(19) United States
(12) Patent Application Publication
(10) Pub. No.: US 2010/0184610 A1
(43) Pub. Date: Jul. 22, 2010

(54) METHOD OF PROGNOSIS

(75) Inventors: Mark William James Ferguson, Manchester (GB); Hugh Gerald Laverty, Manchester (GB); Nicholas Occleston, Manchester (GB); Sharon O'Kane, Manchester (GB); Darren Hodgson, Manchester (GB); Neil French, Manchester (GB); Claire Cridland, Manchester (GB); Philip Roby, Manchester (GB); Ardeshir Bayat, Manchester (GB)

Correspondence Address: BAKER & DANIELS LLP, 300 NORTH MERIDIAN STREET, SUITE 2700, INDIANAPOLIS, IN, 46204, US

(73) Assignee: Renovo Limited, Manchester (GB)

(21) Appl. No.: 12/439,350

(22) PCT Filed: Aug. 28, 2007
(86) PCT No.: PCT/GB2007/003242

§ 371 Date: Jul. 28, 2009

(30) Foreign Application Priority Data
Aug. 31, 2006 (GB) 0617119.3

(51) Int. Cl. C40B 30/04; C12Q 1/68; C40B 40/06; C40B 40/10

(52) U.S. Cl. 506/9; 435/6; 506/16; 506/18

(57) ABSTRACT

Provided are methods, kits and arrays for use in determining susceptibility to keloid formation. These determine susceptibility based on comparison of gene expression in a patient of interest with expression in a control sample. If expression of at least one gene, selected from the group of genes set out in Table 1, is decreased in a sample representative of gene expression in the patient compared to expression of the same gene (or genes) in the control sample this is indicative of a susceptibility to keloid formation.

The invention relates to a method for determining susceptibility to keloid formation. The invention also provides kits and oligonucleotide arrays suitable for use in determining susceptibility to keloid formation.

Keloids (also referred to as keloid scars) are pathological scars produced by an aberrant and over-exuberant wound healing response. Keloids comprise raised scars that spread beyond the margins of an original wound and invade the normal skin surrounding the wound site. Keloids continue to grow over time, and do not regress spontaneously.

Keloids occur with equal frequency in men and women. The incidence of keloid formation is increased in those aged between 10 and 30 years. Keloids may arise as a result of a wide range of injuries, including piercing, surgery, vaccination, tattoos, bites, blunt trauma and burns.

Keloids may have a “domed”, nodular or ridged appearance. Keloids may have a colour similar to that of the surrounding unwounded skin, but are frequently somewhat darker, with a red, purple or brown appearance. Such colour mismatches may increase the visual prominence of keloids. The tendency for hyperpigmentation in keloids is increased on their exposure to solar ultraviolet radiation.

A keloid lesion may be considered to be made up of a number of different portions that may each exhibit quite different biological activity from one another. The central part of a mature keloid lesion (the intra-lesional portion) is largely acellular, while the peripheral part of the lesion (the peri-lesional portion) is relatively more cellular and is the site of increased angiogenic activity. This increase in new blood vessel formation has been linked with the outward growth of the lesion.

Although they represent examples of pathological scarring, keloids are primarily composed of the same cell types and extracellular matrix components that are found in undamaged skin and normal dermal scars. However, the relative abundance and arrangement of these cell types and extracellular matrix components differ from those found in either unwounded skin or normal dermal scars.

The major constituent of keloids is the extracellular matrix component collagen I. Fibroblasts derived from keloids exhibit up to a twenty-fold higher expression of collagen I in vitro, as compared to normal dermal fibroblasts. Similarly, cultured keloid fibroblasts also express elevated levels of elastin and proteoglycans, and it is believed that this increase in extracellular matrix deposition may play a role in keloid development and maintenance.

Collagen I present in keloids is arranged primarily in the form of thick “whorls”, which may be differentiated from the arrangement found in unwounded skin (a so-called “basket weave” of fibrils) and in normal scars (which contain collagen fibres that are thinner than those found in keloids and are arranged approximately parallel to one another). The frequent presence of thickened hyalinized collagen within keloids has led to this form of collagen being termed “keloidal collagen”.

Keloids contain fewer macrophages than do normal scars, but contain abundant eosinophils, mast cells, plasma cells and lymphocytes.

Keloids are seldom a direct cause of pain, but may give rise to discomfort, tenderness, irritation or itching during their formation or growth. Keloids may also impair mechanical function through their size or their increased stiffness compared to unwounded skin. This impairment may be particularly noticeable in the case of keloids located near a joint. Furthermore, it is well recognised that keloids, and in particular large or noticeably disfiguring examples, can cause psychological distress to those afflicted.

A further highly damaging property of keloids is their propensity to recur, particularly following surgical excision. Recurrence of keloids under such circumstances is normally also associated with further expansion of the lesion, and keloids may expand more aggressively following an earlier excision.

Treatment options for hypertrophic scars are similar to those for keloids with the exception that surgical excision is an acceptable and often more favourable approach. Correct clinical treatment of keloids should take into consideration the peculiar difficulties associated with keloid treatment.

Current treatment regimes for keloids include corticosteroid injections, cryotherapy, radiation therapy, silicone gel dressings and intra-lesional injection of agents intended to reduce the size of keloid scarring. A prognosis that a tissue of interest is at risk of keloid formation may, however, be of primary benefit in allowing the avoidance of unsuitable regimes such as surgical excision.

Given their high incidence of recurrence, and the fact that such recurrence is exacerbated by surgical intervention, it is important to be able to accurately determine a risk of keloid formation in order that suitable treatment regimes may be employed from the earliest possible time. Even more preferable may be to avoid elective trauma to individuals at elevated risk of keloid formation. However, although it is recognised that keloid formation is more prevalent in dark skinned races, the causes underlying keloid formation remain unknown and there is a well recognised need for methods and kits able to identify patients susceptible to keloid formation.

Rapid and accurate methods and kits for the determination of susceptibility to keloid formation will facilitate the taking of correct decisions regarding the clinical treatment of those prone to keloid formation. In the case of patients with elevated risk of keloid formation it will be possible to avoid treatments that may initiate keloid development, while such considerations will not be inappropriately applied in the treatment of patients unlikely to form keloids. Importantly, it will be possible to ensure that patients with a susceptibility to keloid formation are provided with preventative or palliative measures at the earliest possible time-points after trauma. The ability to differentiate between keloid-forming and non-keloid-forming patients may be of greatest advantage in terms of limiting surgery, and hence the risk of keloid formation, amongst those prone to keloid development.

It is an aim of certain embodiments of the invention to provide novel methods and kits for determining whether an individual patient or wound has susceptibility to keloid formation. It is another aim of certain embodiments of the present invention to provide methods for determining susceptibility to keloid formation that allow a greater degree of certainty in such determination than may be achieved by the prior art. It is another aim of certain embodiments of the invention to provide methods for determining susceptibility to keloid formation that allow greater speed of determining prognosis than do the methods of the prior art.

In a first aspect of the invention there is provided a method for determining susceptibility to keloid formation, the method comprising:

comparing expression, in a sample representative of gene expression in a patient, of at least one gene, selected from the group of genes set out in Table 1, with expression of the said at least one gene in a control sample;
wherein decreased expression of said at least one gene in the sample representative of gene expression in the patient compared to expression of said at least one gene in the control sample indicates that the patient is susceptible to keloid formation.

In a second aspect of the invention there is provided a kit for determining susceptibility of a patient to keloid formation, the kit comprising:

i) at least one probe capable of binding specifically to a target molecule representative of expression in the patient of at least one gene selected from the group set out in Table 1; and
ii) reference material able to indicate the level of expression of said at least one gene in control tissue.

It is preferred that the methods and kits of the invention to be used for in vitro determination of a patient's susceptibility to keloid formation.

Although the methods and kits of the invention are most suitable for use in association with human patients at risk of keloid formation they may also be useful in determining susceptibility to other similar conditions in non-human animals, such as “proud flesh” in horses.

The present invention is based on the identification by the inventors of a number of genes the decreased expression of which is indicative of an increased susceptibility to keloid formation. The inventors have found that comparison of the expression of one or more of these genes in a sample which is representative of gene expression in a patient with the expression occurring in a control tissue allows an accurate determination of the patient's susceptibility to keloid formation. Increased susceptibility to keloid formation is indicated by a decrease in expression in the patient as compared to that in the control sample, whereas unchanged or increased expression in the patient compared to that in the control indicates that the patient is not susceptible to keloid formation.

The finding that decreased expression of the genes identified in Table 1 (i.e. the group comprising Gene Identification No. 1 to Gene Identification No. 55) may be used in determining the susceptibility of a patient to keloid formation is surprising, since although the expression of certain genes (such as those encoding VEGF, IGF1 and PAI1) has been linked to keloid tissue, the genes set out in Table 1 had never previously been identified as being associated with increased risk of keloid development.

In practicing the invention (whether by use of the methods, kits or arrays of the invention), expression of a selected gene (or genes) in a sample representative of gene expression in the patient is compared with expression of the same gene (or genes) in a suitable control tissue. This comparison of expression of the selected gene (or genes) enables the patient's susceptibility to keloid formation to be determined. If there is decreased expression of the selected gene (or genes) in the sample representative of gene expression in the patient, as compared to in the control sample, then this indicates that the patient is at elevated risk of keloid formation. If, on the other hand, there is no decrease in expression of the selected gene (or genes) in the sample representative of expression by the patient (or, indeed, if there is an increase in expression of these genes), this indicates that the patient does not have a predisposition to keloid formation.

In general expression of selected genes by the patient will be investigated by analysis of target molecules representative of gene expression. Suitable investigation may involve the analysis for presence or absence of such target molecules in a sample (qualitative analysis of gene expression, as discussed further elsewhere in the specification), or analysis of the relative abundance of target molecules in a sample (which may provide quantitative information as to gene expression, as considered in more detail elsewhere in the specification).

Gene expression in the control tissue may be represented by tissues or tissue extracts containing suitable target molecules, or may alternatively be represented by data setting out details of the gene expression levels in the control. The identification, isolation and analysis of suitable target molecules is discussed further elsewhere in the specification, as is the provision of information representative of gene expression in control tissue samples.

Although the inventors have found that any of the genes represented by the group of genes set out in Table 1 may be used in accordance with the present invention, the inventors have further found that certain subsets of these genes have particular prognostic value. These subsets are identified and considered in more detail below.

For example, it is a preferred embodiment of the invention to compare the expression, in the sample representative of gene expression in the patient of at least one gene selected from the group of genes set out in Table 2, with expression of the same gene (or genes) in the control sample. These genes represent a preferred group since the inventors have found that the magnitude of the change in expression shown by these genes is useful in the determination of susceptibility to keloid formation.

It is a more preferred embodiment of the invention to compare the expression, in the sample representative of gene expression in the patient, of at least one gene selected from the group of genes set out in Table 3, with expression of the same gene (or genes) in the control sample. These genes represent a particularly preferred group since the inventors have found that the magnitude of the change in expression shown by these genes is particularly useful in the determination of susceptibility to keloid formation.

Determination of susceptibility to keloid formation in accordance with the present invention may be effected by comparing the expression in a sample representative of gene expression in a patient with expression in a control sample of one gene selected from Table 1, however, it is preferred to utilise multiple genes from Table 1. Thus it may be preferred that determination of susceptibility in accordance with the present invention may be effected by comparing the expression of up to five genes selected from Table 1. It is particularly preferred that determination of susceptibility in accordance with the present invention is effected by comparing the expression of 5, 6, 7, 8, 9 or 10 genes selected from Table 1. Determination of susceptibility to keloid formation in accordance with the present invention may be effected by comparing the expression of up to 15, 20, 30, 40 or even up to 50, genes selected from Table 1. It is most preferred that a determination of susceptibility to keloid formation in accordance with the present invention is effected by comparing the expression of 50 or more genes selected from Table 1. If so desired a determination of susceptibility to keloid formation may be effected using all 55 of the genes identified in Table 1.

It will be appreciated that any individual may constitute a suitable patient able to draw benefit from the methods and kits of the invention, however preferred patients may comprise individuals believed to be at elevated risk of keloid formation. Examples of such individuals include patients with a history of keloid formation, individuals from the African Continental Ancestry Group and individuals from the Asian Continental Ancestry Group.

Suitable patients may include individuals who have experienced, are experiencing or will experience injury to the skin. In particular these may include individuals suffering injury at a site where there is an elevated risk of keloid formation. Examples of such sites may typically include areas of high skin tension, such as the chest, back, shoulders, or neck. However, relevant sites may also include areas, such as the earlobes, that are common sites of keloid formation, although not subject to high skin tension.

The prognostic use of the methods, kits, and arrays of the invention may be useful to patients who have experience, are experiencing, or will experience skin wounding, as well as to patients who have experienced, are experiencing, or will experience skin trauma.

For the purposes of the present invention “skin wounding” may be considered to comprise conditions or clinical situations in which partial or total penetration of the skin occurs, and also those in which partial or total destruction of one or more layers of the skin occurs. For example, wounds may include puncture wounds, incisional wounds, excisional wounds and partial or full thickness skin grafts (including both donor and recipient sites). Such wounds may be associated with surgical procedures or accidental injuries. Wounds may also include burn or scald injuries, resulting from exposure of the skin to substances at high or low temperatures sufficient to cause damage to the skin. Chemical “burns”, such as those caused by exposure of the skin to acid or alkali, may also constitute wounds that may be advantageously assessed for their susceptibility to keloid formation, in accordance with the present invention.

Examples of individuals who are soon to suffer injury such as skin wounding will include those intending to undergo elective surgical procedures; those intending to undergo piercing; those intending to undergo tattooing; and those intending to undergo cosmetic procedures such as dermabrasion or exfoliation (including so-called “chemical peels” and “laser peels”).

For the purposes of the present invention “skin trauma” may be taken as referring to injuries that damage, but do not penetrate, the skin. Illustrative examples of injuries that may be considered as skin trauma include crush injuries to the skin, as well other “blunt” injuries.

Samples representative of gene expression in a patient that may be used in accordance with the present invention encompass any sample that may provide information as to genes being expressed by the patient.

Examples of suitable samples include biopsies, blood samples, urine samples, sputum samples, cerebrospinal fluid samples, and swabbed samples (such as saliva swab samples). Preferred samples include samples of wound tissue, wound fluid, wound aspirates or wound exudates, any of which may enable determination of the susceptibility to keloid formation of the wound from which the sample in question is derived.

In the case of samples derived from wounds, these may be collected at the time of wounding, or at any time following the initial wounding insult. Preferably such samples may be collected within twelve months following the initial wounding insult. More preferably, such samples may be collected within six months following the initial wounding insult, and even more preferably within one month following the initial wounding insult. Most preferably suitable samples derived from wounds may be collected up to seven days following the initial wounding insult.

Suitable samples may be derived from any body site. However, preferred sites from which samples may be derived include those body sites known to be particularly susceptible to keloid formation, for example the shoulders, chest, earlobes, upper arms and cheeks.

Suitable samples may include tissue sections such as histological or frozen sections. Methods by which such sections may be prepared in such a way as to be able to provide information representative of gene expression in the patient from which the section is derived will be well known to those skilled in the art, and should be selected with reference to the technique that it is intended to use when investigating gene expression.

It will be appreciated that suitable samples for use in the methods of the invention may include biopsies derived from a tissue of interest, particularly a tissue believed likely to have an elevated risk of developing into a keloid. Preferably such biopsies may be of a sort selected to reduce the level of injury inflicted to the patient, and thereby limit damage to those found to have increased susceptibility to keloid formation. Such techniques may, for example, make use of needle biopsies in order to reduce the level of injury occurring.

Suitable samples for investigation may include tissues that have been excised during surgical procedures. Such procedures may include scar revision, excision of moles, or excision of benign or malignant tumours. In such cases investigation of the tissue removed will be of great value in determining the patient's risk of keloid formation, and hence a suitable strategy for clinical management of the excision site.

Although the use of samples comprising a portion of tissue from the patient is contemplated, it may generally be preferred that the sample representative of gene expression comprise a suitable extract taken from such a tissue, said extract being capable of investigation to provide information regarding gene expression in the patient. Suitable protocols which may be used for the production of tissue extracts capable of providing information regarding gene expression in a patient will be well known to those skilled in the art. Preferred protocols may be selected with reference to the manner in which gene expression is to be investigated. Illustrative examples of protocols that may be used to produce tissue extracts representative of gene expression in a patient are discussed below.

Suitable control samples, for use in accordance with methods or kits of the invention, may be selected with reference to the source of the sample representative of gene expression in the patient. Sources and examples of suitable control samples will be apparent to those skilled in the art and include those derived from individuals that are not subject to keloid formation. It will be recognised that the skin constitutes a preferred source of both patient and control samples.

Suitable control samples may include portions of non-keloid tissues or organs including target molecules representative of gene expression (in which case the tissue should be preserved in such a manner that information regarding the expression of genes in the tissue may be extracted from the tissue, for example by analysis of the target molecules). Alternatively, suitable control samples may comprise tissue extracts incorporating extracted and/or isolated target molecules (such as mRNA or cDNA) that are representative of gene expression in the control sample. Relevant information regarding gene expression in control samples may also be provided in the form of data derived from such samples, as considered elsewhere in the specification.

Control samples from which information relating to the expression of selected genes may be derived include tissue samples and tissue extracts as considered herein with reference to patient samples. For example, such information may be derived directly from a tissue or organ sample constituting the control sample, or from an extract capable of providing information regarding gene expression in the selected control sample. The expression of the selected gene, or genes, (selected from the group of genes set out in Table 1) in control samples of this type may be investigated using the methods described herein in connection with the investigation of gene expression in the patient.

Although tissue or organ samples constituting control samples, or extracts from such samples, may be used directly as the source of information regarding gene expression in the control sample (as discussed elsewhere in the specification), it will generally be preferred that information regarding the expression of the selected gene (or genes) in the control sample be provided in the form of reference data. Such reference data may be provided in the form of tables indicative of gene expression in the chosen control tissue. Alternatively, the reference data may be supplied in the form of computer software containing retrievable information indicative of gene expression in the chosen control tissue. The reference data may, for example, be provided in the form of an algorithm enabling comparison of expression of at least one selected gene (or genes) in the patient with expression of the same gene (or genes) in the control tissue sample.

In a preferred embodiment of the invention, a prognostic result indicative of a patient's susceptibility to keloid formation may be delivered automatically on inputting results representative of expression of selected genes in the patient's sample into a predictive algorithm that has been trained upon data representative of gene expression in a suitable control sample. Well-established and commonly used classification systems include, but are not limited to, K-Nearest Neighbours, Centroid Classification, Linear Discriminant Analysis, Neural Networks and Support Vector Machines available, for example, in the Partek Genomics Suite software package (Partek Inc.).

A suitable sample representative of gene expression in a patient or control sample may provide qualitatitive and/or quantitative information regarding gene expression. For the purpose of the present invention qualitative information regarding gene expression is to be considered to be information that provides identification as to genes expressed in a patient or control sample, without providing information as to the relative amounts of expression (save as to whether a particular gene is, or is not, expressed). It will be appreciated that in some situations qualitative information may allow a sufficient comparison between expression in the patient and the control sample to allow a determination of the risk of keloid formation. Qualitative information may be particularly suitable for determinations of susceptibility that are based on decreased expression of genes of Table 1 that are normally expressed in control samples, but are not expressed at all in patients at increased risk of keloid formation. In such cases the lack of expression of the gene by a patient will be sufficient to indicate an elevated risk of keloid formation. Examples include Jumonji Domain Containing 2A (Gene Identification No. 17) and HGFL (Gene Identification No. 21), and it may be a preferred embodiment of the invention to investigate expression of one or both of these genes.

It will, however, generally be preferred to use a sample capable of providing quantitative information regarding gene expression in the patient or control sample. Such information allows ready comparison between the levels of expression in the patient and the levels of expression in the control sample. For the purposes of the present invention quantitative information relating to gene expression may be taken to refer to either absolute or relative quantification. Methods by which absolute or relative quantitation may be achieved are discussed further below.

Samples representative of gene expression in patient or control samples will generally contain target molecules that are directly or indirectly representative of gene expression. Suitable samples may be provided in the form of tissue samples containing such target molecules, or, preferably as tissue extracts. A tissue extract representative of gene expression in a patient will generally contain isolated target molecules that are representative of gene expression in the tissue from which the extract is obtained.

Suitable techniques by which tissue samples or tissue extracts may be obtained and prepared in order that they may provide information as to gene expression may be selected with reference to the type of target molecule that is to be employed. Examples of appropriate techniques that may be used will be readily apparent to the skilled person, however guidance as to suitable techniques is also provided elsewhere in the specification.

It will be appreciated that protein target molecules represent target molecules that are particularly amenable to direct detection. Such direct detection may provide qualitative or quantitative information as to the amount of the protein present in the patient or control sample, thereby allowing comparison of expression.

In a preferred instance, the amount of certain target proteins present in a sample may also be assessed with reference to the biological activity of the target in the sample. Assessment and comparison of expression in this manner is particularly suitable in the case of protein targets having enzyme activity. Examples of genes set out in Table 1 having enzyme activity, and so particularly suitable for investigation in this manner, include those identified by Gene Identification Numbers 3, 15, 18, 20, 33, 35, 44 and 55. Enzyme activity of protein targets may, for example, be investigated by analysing breakdown of labelled enzyme substrate, and the amount of enzyme activity thereby correlated with gene expression occurring in the patient or control sample.

The presence or absence of target molecules in a tissue sample or extract will generally be detected using suitable probe molecules (although there may be some instances, such as those discussed above, where presence or absence of a target molecule may be determined directly without the need for a probe). Such detection will provide information as to gene expression, and thereby allow comparison between gene expression occurring in the patient and expression occurring in the control sample.

Probes will generally be capable of binding specifically to target molecules directly or indirectly representative of gene expression in the patient or control sample. Binding of such probes may then be assessed and correlated with gene expression to allow an effective prognostic comparison between gene expression in the patient and in the control. Suitable probes that may be used in the methods, kits and arrays of the invention are discussed elsewhere in the specification.

Target molecules suitable for use in the methods, kits and arrays of the invention are molecules representative of gene expression either directly or indirectly, as considered in greater detail below. Target molecules may include mRNA gene transcripts, as well as natural and artificial products of such transcripts (e.g. proteins or cDNA respectively). It will be appreciated that samples for use in accordance with the present invention should be processed in a manner selected with reference to the nature of the target molecule that is to be used. Suitable protocols for processing of tissues to yield samples containing usable target molecules are discussed further below.

Suitable target molecules may comprise the direct products of gene expression. Such direct products of gene expression may, for example, comprise one or more gene transcripts representative of gene expression. The use of mRNA gene transcripts as target molecules allowing comparison of gene expression in the patient with expression in the control sample is a preferred embodiment of the invention.

Alternatively, a sample representative of gene expression in the patient or control sample may comprise target molecules that are indirectly representative of gene expression. Examples of such targets indirectly representative of gene expression may include natural products (such as proteins) that are produced on translation of a gene transcript, as well as artificial products generated from gene transcripts. Preferred examples of artificial target molecules generated from gene transcripts include cDNA and cRNA, either of which may be generated using well known protocols or commercially available kits or reagents.

For example, in a preferred embodiment, RNA representative of gene expression in a patient or control sample may be isolated through a process of lysing cells taken from a suitable sample (which may be achieved using a commercially available lysis buffer such as that produced by Qiagen Ltd.) followed by centrifugation of the lysate using a commercially available nucleic acid separation column (such as the RNeasy midi spin column produced by Qiagen Ltd). Other methods for RNA extraction include variations on the phenol and guanidine isothiocyanate method of Chomczynski, P. and Sacchi, N. (1987) Analytical Biochemistry 162, 156. “Single Step Method of RNA Isolation by Acid Guanidinium Thiocyanate-Phenol-Chloroform Extraction.” RNA obtained in this manner may constitute a suitable target molecule itself, or may serve as a template for the production of target molecules representative of gene expression.

It may be preferred that RNA derived from a patient or control sample may be used as substrate for cDNA synthesis, for example using the Superscript System (Invitrogen Corp.). The resulting cDNA may then be converted to biotinylated cRNA using the BioArray RNA Transcript labelling Kit (Enzo Life Sciences Inc.) and this cRNA purified from the reaction mixture using an RNeasy mini kit (Qiagen Ltd).

In the case of protein target molecules, gene expression may be assessed with reference to the total amount of the protein target present. Suitable techniques for the measurement of the amount of a protein target present in a sample representative of gene expression in a patient or control sample include, but are not limited to, aptamers and antibody-based techniques, such as radio-immunoassays (RIAs), enzyme-linked immunoassays (ELISAs) and Western blotting, immuno-PCR and multiplex approaches such as those using beads or microspheres (for example xMap technology from Luminex Inc), (Bloom and Dean (2003) Biomarkers in Clinical Drug Development; Crowther (1995) Elisa Theory and Practice (Humana Press); Singh et al (1993) Diagnostics in the year 2000: Antibody, Biosensor and nucleic acid Technologies (Van Nostrand Reinhold, N.Y.); Niemeyer C M, Adler M, Wacker R. Immuno-PCR: high sensitivity detection of proteins by nucleic acid amplification. Trends Biotechnol. 2005 April; 23(4):208-16; Abreu I, Laroche P, Bastos A, Issert V, Cruz M, Nero P, Fonseca J E, Branco J, Machado Caetano J A. Multiplexed immunoassay for detection of rheumatoid factors by FIDISTM technology. Ann N Y Acad Sci. 2005 June; 1050:357-63).

The disclosures of the documents set out in the preceding paragraphs are incorporated by reference, insofar as they describe methods that may useful to the skilled person in practising the present invention.

In the event that expression of one or more genes from Table 1 in a control sample is to be investigated via processing of a tissue or organ sample constituting the control sample, or by processing of a tissue extract representative of gene expression in the control sample, for example to isolate suitable target molecules, it is preferred that such processing is conducted using the same methods used to process the sample from the patient. Such parallel processing of patient samples and control samples allows a greater degree of confidence that comparisons of gene expression in these tissues will be normalised relative to one another (since any artefacts associated with the selected method by which tissue is processed and gene expression investigated will be applied to both the patient and control samples).

Furthermore, the parallel processing of the control sample in this manner provides an “internal control” that will allow the practitioner to confirm that processing has occurred successfully. Since the practitioner will be aware that the selected one or more genes from Table 1 that have been selected for comparison of expression are normally expressed by control tissues, the practitioner will be able to discount any instances of processing (for investigation of gene expression) which give rise to assays indicating that expression of these internal controls cannot be detected (since these results will likely be as a result of a processing error leading to artificially low readings). Such results may otherwise give rise to an incorrect assessment that the patient is susceptible to keloid formation (since the same artificial decrease in assessed expression would be noted in respect of the selected gene or genes from Table 1).

Samples representative of gene expression in a patient, or a control tissue, may be manipulated prior to effecting comparison of gene expression. Such manipulation may, for example, be designed to make comparison of expression easier, or to increase the information made available by the comparison. Examples of suitable ways in which such samples may be manipulated are considered below.

Preferably the methods or kits of the invention will provide means by which the expression data relating to the patient and control tissue may be “normalised” with respect to one another. Normalisation ensures that comparisons being made are “like for like”, and suitable parameters for use in normalisation are well known to those skilled in the art. Purely by way of illustration, normalisation may be effected with reference to cell numbers in the samples to be compared; and/or total protein content of samples to be compared; and/or total nucleic acid content of samples to be compared; and/or expression level of one or more genes the expression of which does not change between keloid-forming and non-keloid-forming tissues. Alternatively or additionally, a suitable control may involve assessing expression of one or more genes known to be expressed in keloids. Detection of the expression of such genes (in combination with the reduced expression of one or more of the genes set out in Table 1) will provide a suitable control against which gene expression can be referenced. Suitable examples of such genes are considered elsewhere in the specification.

The inventors have found that preferred samples representative of gene expression for use in accordance with the present invention are those samples comprising nucleic acid target molecules representative of gene expression. For the purposes of the present invention a nucleic acid target is a nucleic acid the presence or absence of which is to be detected, or the amount of which present is to be quantified. Such detection or quantification will allow a prognostic comparison of expression to be effected. A target nucleic acid may preferably have a sequence that is complementary to the nucleic acid sequence of a corresponding probe directed to the target. A nucleic acid target in accordance with the present invention may encompass both a specific subsequence of a larger nucleic acid to which a probe is directed or, alternatively, the overall sequence (e.g. complete mRNA transcript) whose expression level it is desired to detect. Suitable nucleic acid targets may include both RNAs and DNAs, and encompass both naturally occurring and artificial nucleic acids.

It will be understood that target nucleic acids suitable for use in accordance with the invention need not comprise “full length” nucleic acids (e.g. full length gene transcripts), but need merely comprise a sufficient length to allow specific binding of probe molecules.

It will be understood that “nucleic acids” or “nucleic acid molecules” for the purposes of the present invention refer to a deoxyribonucleotide or ribonucleotide polymers in either single- or double-stranded form. Furthermore, unless the context requires otherwise, these terms should be taken to encompass known analogues of natural nucleotides that can function in a similar manner to naturally occurring nucleotides.

mRNA constitutes a preferred form of target molecule that may be used in the methods and kits of the invention. mRNA gene transcripts are directly representative of gene expression in the patient or control sample.

It will be recognised that mRNA, representative of gene expression, may be found directly in a tissue derived from a patient or control sample, without the need for mRNA extraction or purification. For example, mRNA present in, and representative of gene expression in, a patient or control sample of interest may be investigated using appropriately fixed sections or biopsies of such a tissue. The use of samples of this kind may provide benefits in terms of the rapidity with which comparisons of expression can be made, as well as the relatively cheap and simple tissue processing that may be used to produce the sample. In situ hybridisation techniques represent preferred methods by which gene expression may be investigated and compared in tissue samples of this kind. Techniques for the processing of tissues of interest that maintain the availability of RNA representative of gene expression in the patient or control sample are well known to those of skill in the art.

However, techniques by which mRNAs representative of gene expression in a patient or control sample may be extracted and collected are also well known to those skilled in the art, and the inventors have found that such techniques may be advantageously employed in accordance with the present invention. Samples comprising extracted mRNA from a patient or control sample may be preferred for use in the methods and kits of the invention, since such extracts tend to be more readily investigated than is the case for samples comprising the original tissues. For example, suitable target molecules allowing for comparison of gene expression may comprise the total RNA isolated from a sample of tissue from the patient, or a sample of control tissue.

Furthermore, extracted RNA may be readily amplified to produce an enlarged mRNA sample capable of yielding increased information on gene expression in the patient or control sample. Suitable examples of techniques for the extraction and amplification of mRNA populations are well known, and are considered in more detail below.

By way of example, methods of isolation and purification of nucleic acids to produce nucleic acid targets suitable for use in accordance with the invention are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993).

In a preferred method, the total nucleic acid may be isolated from a given sample using, for example, an acid guanidinium-phenol-chloroform extraction method.

In the event that it is desired to amplify the nucleic acid targets prior to investigation and comparison of gene expression it may be preferred to use a method that maintains or controls for the relative frequencies of the amplified nucleic acids in the patient or control tissue from which the sample is derived.

Suitable methods of “quantitative” amplification are well known to those of skill in the art. One well known example, quantitative PCR involves simultaneously co-amplifying a control sequence whose quantities are known to be unchanged between control and patient samples. This provides an internal standard that may be used to calibrate the PCR reaction.

In addition to the methods outlined above, the skilled person will appreciate that any technology coupling the amplification of gene-transcript specific product to the generation of a signal may also be suitable for quantitation. A preferred example employs convenient improvements to the polymerase chain reaction (U.S. Pat. Nos. 4,683,195 and 4,683,202) that have rendered it suitable for the exact quantitation of specific mRNA transcripts by incorporating an initial reverse transcription of mRNA to cDNA. Further key improvements enable the measurement of accumulating PCR products in real-time as the reaction progresses. Examples of suitable technologies using fluorescent resonance energy transfer to generate a quantitative gene-specific signal include Taqman (U.S. Pat. Nos. 5,210,015 and 5,487,972), molecular beacons (WO-95/13399) and scorpions (US2005/0164219). The parallel quantitation of multiple transcripts is possible via the use of different fluorescent moieties for each gene target.

Other suitable amplification methods include, but are not limited to Nucleic acid sequence based amplification (NASBA) (Saad F. UPM3: review of a new molecular diagnostic urine test for prostate cancer. Can J. Urol. 2005 February; 12 Suppl 1:40-3); Rolling Circle Amplification (RCA) (Gomez K F, Lane J, Cunnick G, Grimshaw D, Jiang W G, Mansel RE. From PCR to RCA: a surgical trainee's guide to the techniques of genetic amplification. Eur J Surg Oncol. 2002 August; 28(5):554-9); Branched Chain Nucleic Acids (BCNA) (Andras S C, Power J B, Cocking E C, Davey M R. Strategies for signal amplification in nucleic acid detection. Mol Biotechnol. 2001 September; 19(1):29-44); the invader assay (de Arruda M, Lyamichev V I, Eis P S, Iszczyszyn W, Kwiatkowski R W, Law S M, Olson M C, Rasmussen E B. Invader technology for DNA and RNA analysis: principles and applications. Expert Rev Mol Diagn. 2002 September; 2(5):487-96); ligase chain reaction (LCR) (see Wu and Wallace, Genomics, 4: 560 (1989), Landegren, et al., Science, 241: 1077 (1988) and Barringer, et al., Gene, 89: 117 (1990); transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

In a particularly preferred embodiment, the mRNA transcripts from a tissue representative of gene expression in a patient or control sample may be reverse transcribed with a reverse transcriptase and a promoter consisting of oligo dT and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook, supra.) and this particular method is described in detail by Van Gelder, et al., Proc. Natl. Acad. Sci. USA, 87: 1663-1667 (1990) who demonstrate that in vitro amplification according to this method preserves the relative frequencies of the various RNA transcripts. Moreover, Eberwine et al. Proc. Natl. Acad. Sci. USA, 89: 3010-3014 (1992) provide a protocol that uses two rounds of amplification via in vitro transcription to achieve greater than 10⁶ fold amplification of the original starting material, thereby permitting expression monitoring even when only a small sample of the tissue of interest is available.

It will be appreciated by one of skill in the art that the direct transcription method described above leads to the production of antisense RNA (aRNA) targets. In such cases probes, such as oligonucleotide probes, to be used to investigate and compare gene expression should be chosen to be complementary to sequences or sub-sequences of the antisense nucleic acids.

The skilled person will further appreciate that artificial nucleic acid molecules may also be used in the comparison of gene expression. Examples of artificial target molecules suitable for use in accordance with the present invention include cDNAs made by reverse transcription of mRNA, or second strand cDNA or RNA (cRNA) transcribed from a double stranded cDNA intermediate. Methods for the production of cDNAs and cRNAs are well documented in the art, and will be known to the skilled person, and indeed kits and reagents suitable for their production are commercially available.

For the purposes of the present invention, a sample that is “representative” of gene expression in a patient is to be considered to encompass any sample providing information as to the expression of genes in the tissues of the patient. For example, a representative sample may provide information as to all the genes expressed by the patient, and preferably the relative levels of expression of said genes.

In a preferred embodiment, a representative sample is one in which the concentration of target molecules is proportional to the concentration of mRNA gene transcripts of the gene or genes, the expression of which, by the patient, is to be compared to controls. While it is preferred that the proportionality be relatively strict (e.g., a doubling in the number of mRNA gene transcript occurring in the patient leading to a doubling in the number of corresponding target molecules present in the sample), the skilled person will appreciate that the proportionality can be more relaxed and even non-linear. For example, an assay where a five fold difference in concentration of the mRNA gene transcripts in tissue from the patient results in a three to six fold difference in the concentration of target molecules in the representative sample is sufficient for most purposes.

In the event that more precise quantification is required, serial dilutions of “standard” target molecules can be used to prepare calibration curves according to methods well known to those skilled in the art. More preferably quantitation of target molecules will be relative and normalised with respect to each other and/or ‘housekeeping’ genes whose expression levels are not increased in keloid forming as compared to non-keloid forming tissues. Examples of such genes include exportin 7 (XPO7), Cleavage and Polyadenylation Specific Factor 4, 30 kDa (CPSF4), F-box only protein 7 (FBXO7), ADP-ribosylation factor 1 (ARF1), signal sequence receptor beta (SSR2) and methionine-tRNA synthetase (MARS).

It will, of course, be appreciated that in the case of a qualitative sample or samples (where simple detection of the presence or absence of gene expression is desired) no such elaborate control or calibration is required.

Although it may be preferred in many instances that the representative sample provides information as to all genes expressed in the patient or control sample, a suitable representative sample may alternatively provide information relating to the expression of only a sub-set of the total number of genes undergoing expression.

In many cases it may be preferred to assess the degree of gene expression in patient or control samples using probe molecules capable of indicating the presence of target molecules (representative of one or more of the genes set out in Table 1) in the relevant sample.

The use of target molecules and probes in methods, kits or assays in accordance with the present invention may confer increased sensitivity on the methods of the invention. This may lead to an increased ability to discriminate between otherwise small differences between expression in the patient and expression in the control sample.

Generally, suitable probes for use in the present invention will bind to their target molecules, and thereby allow detection of the target molecule (this detection being indicative of expression of the gene selected from Table 1 represented by the target molecule).

It may be preferred that probes for use in accordance with the invention allow replication of the target molecules (suitably in combination with the probe molecule). Replication in this manner produces a greater number of target molecules, and thus allows further binding of the labelled probe. In turn, the increased amount of labelled probe thus bound amplifies the detectable signal indicative of gene expression.

Probes for use in the methods and kits of the invention may be selected with reference to the product (direct or indirect) of gene expression to be investigated. Examples of suitable probes include oligonucleotide probes, antibodies, aptamers, and binding proteins or small molecules having suitable specificity.

Oligonucleotide probes constitute preferred probes suitable for use in accordance with the methods and kits of the invention. The generation of suitable oligonucleotide probes is well known to those skilled in the art (Oligonucleotide synthesis: Methods and Applications, Piet Herdewijn (ed) Humana Press (2004).). Oligonucleotide and modified oligonucleotides are commercially available from numerous companies.

An oligonucleotide is a single-stranded nucleic acid ranging in length from 2 to about 500 nucleotide bases, preferably from about 5 to about 50 nucleotides, more preferably from about 10 to about 40 nucleotides and most preferably from about 15 to about 40 nucleotides in length. Suitable hybridization methods, conditions, times, fluid volumes, and suitable methods by which hybridisation of oligonucleotide probes may be detected are as described elsewhere in the present specification.

For the purposes of the present invention an oligonucleotide probe may be taken to comprise an oligonucleotide capable of hybridising specifically to a target nucleic acid of complementary sequence through one or more types of chemical bond. Such binding may usually occur through complementary base pairing, and usually through hydrogen bond formation. Suitable oligonucleotide probes may include natural (ie., A, G, C, or T) or modified bases (7-deazaguanosine, inosine, etc.). In addition, a linkage other than a phosphodiester bond may be used to join the bases in the oligonucleotide probe(s), so long as this variation does not interfere with hybridisation of the oligonucleotide probe to its target. Thus, oligonucleotide probes suitable for use in the methods and kits of the invention may be peptide nucleic acids in which the constituent bases are joined by peptide bonds rather than phosphodiester linkages.

The phrase “hybridising specifically to” as used herein refers to the binding, duplexing, or hybridising of an oligonucleotide probe preferentially to a particular target nucleotide sequence under stringent conditions when that sequence is present in a complex mixture (such as total cellular DNA or RNA). Preferably a probe may bind, duplex or hybridise only to the particular target molecule.

The term “stringent conditions” refers to conditions under which a probe will hybridise to its target subsequence, but minimally to other sequences. Preferably a probe may hybridise to no sequences other than its target under stringent conditions. Stringent conditions are sequence-dependent and will be different in different circumstances. Longer sequences hybridise specifically at higher temperatures.

In general, stringent conditions may be selected to be about 5° C. lower than the thermal melting point (Tm) for the specific sequence at a defined ionic strength and pH. The Tm is the temperature (under defined ionic strength, pH, and nucleic acid concentration) at which 50% of the oligonucleotide probes complementary to a target nucleic acid hybridise to the target nucleic acid at equilibrium. As the target nucleic acids will generally be present in excess, at Tm, 50% of the probes are occupied at equilibrium. By way of example, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

Considerations for the design and selection of probes suitable for use with antisense nucleic acid targets (aRNA) have been discussed above. In the case that the nucleic acid targets comprise sense nucleic acids, suitable oligonucleotide probes may be selected to be complementary to sequences or sub-sequences of the sense nucleic acids. In the case of nucleic acid targets that are double stranded, suitable probes may be of either sense as the nucleic acid targets will provide both sense and antisense strands.

Antibodies suitable for use in the methods or kits of the invention may be used to detect target molecules, such as proteins, that represent gene expression in a tissue of interest.

Antibodies that may be used to investigate gene expression in accordance with the methods and kits of the present invention include monoclonal antibodies and polyclonal antibodies, as well as fragments of such antibodies, including, but not limited to, Fab or F(ab')hd 2, and Fv fragments.

Methods suitable for the generation and/or identification of antibodies capable of binding specifically to a given target are well known to those skilled in the art. In general suitable antibodies may be generated by the use of the isolated target as an immunogen. This immunogen is administered to a mammalian organism, such as, but not limited to, a rat, rabbit, goat or mouse, and antibodies elicited as part of the immune response. Generally antibodies will be used in the context of the methods and kits of the invention to bind to protein products of gene expression. Suitable immunogens may include the full-length protein to be investigated, or an antigenic peptide fragment thereof.

Monoclonal antibodies can be produced by hybridomas, immortalized cell lines capable of secreting a specific monoclonal antibody. The immortalized cell lines can be created in vitro by fusing two different cell types, usually lymphocytes, one of which is a tumour cell.

Aptamers are nucleic acid molecules that assume a specific, sequence-dependent shape and bind to specific target ligands based on a lock-and-key fit between the aptamer and ligand. Typically, aptamers may comprise either single- or double-stranded DNA molecules (ssDNA or dsDNA) or single-stranded RNA molecules (ssRNA).

Aptamers may be used to bind both nucleic acid and non-nucleic acid targets. Accordingly aptamers are suitable probes for use in the investigation of gene expression products including RNA, DNA and small molecules or proteins. Preferably aptamers may be used to investigate gene expression products having a molecular weight of between 100 and 10,000 Da. ssDNA aptamers may be preferred for use in the investigation of gene expression products comprising DNA.

Suitable aptamers may be selected from random sequence pools, from which specific aptamers may be identified which bind to the selected target molecules with high affinity. Methods for the production and selection of aptamers having desired specificity are well known to those skilled in the art, and include the SELEX (systematic evolution of ligands by exponential enrichment) process. Briefly, large libraries of oligonucleotides are produced, allowing the isolation of large amounts of functional nucleic acids by an iterative process of in vitro selection and subsequent amplification through polymerase chain reaction.

The use of aptamers for investigation of gene expression in accordance with the methods and kits of the invention may be advantageous, since aptamers have relatively stable shelf lives. Aptamers suitable for use in the methods and/or kits of the invention may preferably be stabilized by chemical modifications (for example 2′-NH₂ and 2′-F modifications).

Photoaptamers are a subclass of aptamers incorporating at least one bromo-deoxyuridine (BrdU) in place of a thymidine (T) nucleotide. The presence of the BrdU enables photoaptamers to form a specific covalent crosslink with their target ligands when exposed to ultraviolet light. Because crosslinking requires both affinity-based binding and close proximity between a BrdU (at a specific location in the photoaptamer) and an amino acid (at a specific location in the target ligand), photoaptamers may be preferred for use in the methods and kits of the invention when increased specificity of binding with a gene expression product is required.

Suitable methods by which gene expression may be compared in accordance with the present invention may be selected in the light of the considerations referred to in the preceding pages.

In general, methods for analysis may be selected based on the nature of a target molecule to be investigated, and suitable selection criteria may distinguish between nucleic acid and protein target molecules.

However, as set out above, it may generally be preferred to investigate and compare gene expression using oligonucleotide probes capable of binding to nucleic acid target molecules.

Oligonucleotide probes may be used to detect complementary nucleic acid sequences (i.e., nucleic acid targets) in a suitable representative sample. Such complementary binding forms the basis of most techniques in which oligonucleotides may be used to detect, and thereby allow comparison of, expression of particular genes. Preferred technologies permit the parallel quantitation of the expression of multiple genes and include technologies where amplification and quantitation of species are coupled in real-time, such as the quantitative reverse transcription PCR technologies previously described herein, and technologies where quantitation of amplified species occurs subsequent to amplification, such as array technologies.

Array technologies involve the hybridisation of samples, representative of gene expression within the patient or control sample, with a plurality of oligonucleotide probes wherein each probe preferentially hybridises to a disclosed gene or genes. Array technologies provide for the unique identification of specific oligonucleotide sequences, for example by their physical position (e.g., a grid in a two-dimensional array as commercially provided by Affymetrix Inc.) or by association with another feature (e.g. labelled beads as commercially provided by Illumina Inc or Luminex Inc). Oligonucleotide arrays may be synthesised in situ (e.g by light directed synthesis as commercially provided by Affymetrix Inc) or pre-formed and spotted by contact or ink jet technology (as commercially provided by Agilent or Applied Biosystems). It will be apparent to those skilled in the art that whole or partial cDNA sequences may also serve as probes for array technology (as commercially provided by Clontech).

Oligonucleotide probes may be used in blotting techniques, such as Southern blotting or northern blotting, to detect and compare gene expression (for example by means of cDNA or mRNA target molecules representative of gene expression). Techniques and reagents suitable for use in Southern or northern blotting techniques will be well known to those of skill in the art. Briefly, samples comprising DNA (in the case of Southern blotting) or RNA (in the case of northern blotting) target molecules are separated according to their ability to penetrate a gel of a material such as acrylamide or agarose. Penetration of the gel may be driven by capillary action or by the activity of an electrical field. Once separation of the target molecules has been achieved these molecules are transferred to a thin membrane (typically nylon or nitrocellulose) before being immobilized on the membrane (for example by baking or by ultraviolet radiation). Gene expression may then be detected and compared by hybridisation of oligonucleotide probes to the target molecules bound to the membrane. More details of suitable conditions in which hybridisation may be effected are provided below, as are examples of techniques by which hybridisation may be detected.

In certain circumstances the use of traditional hybridisation protocols for comparing gene expression may prove problematic. For example blotting techniques may have difficulty distinguishing between two or more gene products of approximately the same molecular weight since such similarly sized products are difficult to separate using gels. Accordingly, in such circumstances it may be preferred to compare gene expression using alternative techniques, such as those described below.

Gene expression in a sample representing gene expression in a patient may be assessed with reference to global transcript levels within suitable nucleic acid samples by means of high-density oligonucleotide array technology. Such technologies make use of arrays in which oligonucleotide probes are tethered, for example by covalent attachment, to a solid support. These arrays of oligonucleotide probes immobilized on solid supports represent preferred components to be used in the methods and kits of the invention for the comparison of gene expression. Large numbers of such probes may be attached in this manner to provide arrays suitable for the comparison of expression of large numbers of genes selected from those set out in Table 1. Accordingly it will be recognised that such oligonucleotide arrays may be particularly preferred in embodiments of the methods or kits of the invention where it is desired to compare expression of more than one gene selected from Table 1.

In a preferred embodiment investigation of gene expression using oligonucleotide arrays may be effected by hybridisation of oligonucleotide probes and nucleic acid targets at low stringency followed by at least one wash at higher stringency. Low stringency conditions suitable for use in accordance with these embodiments may comprise a reaction temperature of about 20° C. to about 50° C. (more preferably about 30° C. to about 40° C., and most preferably about 37° C.) and 6×SSPE-T buffer (or lower). Suitable hybridisation protocols may include subsequent washes at progressively increasing stringency until a desired level of hybridisation specificity is reached. Hybridisation stringency may also be varied by electronic means, for example as provided by Nanogen Inc. (Sosnowski R, Heller M J, Tu E, Forster A H, Radtkey R. Active microelectronic array system for DNA hybridization, genotyping and pharmacogenomic applications. Psychiatr Genet. 2002 December; 12(4):181-92).

Suitable techniques for the detection of hybridisation between oligonucleotide probes and nucleic acid targets are considered further below.

The identity of selected olignonucleotide probes incorporated in arrays may be altered to allow more detailed selection of the genes, the expression of which is to be compared. For example arrays suitable for use in the methods or kits of the invention may comprise one or more oligonucleotide probes selected with reference to the differential expression of selected genes from Tables 1 to 3 as considered previously.

Alternatively, assessment of gene expression in a patient or control sample, based on levels of nucleic acids sequences (such as mRNA or DNA) in a sample representative of gene expression in the patient or control, may be undertaken using other suitable techniques that will be apparent to the skilled person. For example, northern blotting provides a sensitive method by which levels of mRNA representative of gene expression in a patient or control sample may be assessed.

Other suitable methodologies that may be used in the comparison of nucleic acid targets representative of gene expression include, but are not limited to, nucleic acid sequence based amplification (NASBA); rolling circle DNA amplification (RCA); branched chain nucleic acid and invader assays; the use of aptamers, antibodies or antibody derivatives (Singh et al, 1993; Boeckh and Boivin 1998; Bloom and Dean, 2003; Jain, 2004; Millar and Moore, 2004; Olson, 2004; Yang and Rothman, 2004).

As described previously, gene expression in a patient or control sample may alternatively be investigated using samples comprising proteins representative of gene expression. Suitable techniques by which such protein samples may be investigated to assess gene expression include, but are not limited to, aptamer detection; mass spectrometry; nuclear magnetic resonance (NMR); antibody-based methods such as immuno-PCR and multiplex approaches such as those using arrays, beads or microspheres (for example xMap technology from Luminex Inc), ELISA, RIA and Western blotting; and other methods well known to those skilled in the art (Bloom and Dean (2003) Biomarkers in Clinical Drug Development; Crowther (1995) Elisa Theory and Practice (Humana Press); Singh et al (1993) Diagnostics in the year 2000: Antibody, Biosensor and nucleic acid Technologies (Van Nostrand Reinhold, N.Y.); Niemeyer C M, Adler M, Wacker R. Immuno-PCR: high sensitivity detection of proteins by nucleic acid amplification. Trends Biotechnol. 2005 April; 23(4):208-16; Abreu I, Laroche P, Bastos A, Issert V, Cruz M, Nero P, Fonseca J E, Branco J, Machado Caetano J A. Multiplexed immunoassay for detection of rheumatoid factors by FIDISTM technology. Ann N Y Acad Sci. 2005 June; 1050:357-63).

For instance, expression of proteins having enzymatic activity may be investigated and compared using assays based around activity of the protein in question. Enzymatic protein extracts (here constituting samples representative of gene expression in the patient or control sample) may, for example, be incubated with samples comprising known quantities of an appropriately labelled substrate. The amount of enzymatic activity, and hence an indication of the level of gene expression in the patient or control sample, may be determined by the amount of substrate converted by the enzyme.

Detection of probe or target molecules can be facilitated by coupling (i.e., physical linking) of such molecules to a detectable moiety. Alternatively suitable probe or target molecules may be synthesised such that they incorporate detectable moieties. Techniques that may be used in the coupling or incorporation of detectable moieties in probe or target molecules suitable for use in the method, kits or arrays of the invention are considered below.

Examples of detectable moieties that may be used in the labelling of probes or targets suitable for use in accordance with the invention include any composition detectable by spectroscopic, photochemical, biochemical, immunochemical, electrical, optical or chemical means. Suitable detectable moieties include various enzymes, prosthetic groups, fluorescent materials, luminescent materials, bioluminescent materials, radioactive materials and colourimetric materials. These detectable moieties are suitable for incorporation in all types of probes or targets that may be used in the methods or kits of the invention unless indicated to the contrary.

Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, phycoerythrin, texas red, rhodamine, green fluorescent protein, and the like; an example of a luminescent material includes luminol; examples of bioluminescent materials include luciferase, luciferin, and aequorin; examples of suitable radioactive material include ¹²⁵I, ¹³¹I, ³⁵S, ³H, ¹⁴C, or ³²P; examples of suitable colorimetric materials include colloidal gold or coloured glass or plastic (e.g., polystyrene, polypropylene, latex, etc.) beads.

Means of detecting such labels are well known to the skilled person. For example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the coloured label.

In a preferred embodiment of the invention fluorescently labelled probes or targets may be scanned and fluorescence detected using a laser confocal scanner.

In the case of labelled nucleic acid probes or targets suitable labelling may take place before, during, or after hybridisation. In a preferred embodiment, nucleic acid probes or targets for use in the methods or kits of the invention are labelled before hybridisation. Fluorescence labels are particularly preferred and, where used, quantification of the hybridisation of the nucleic acid probes to their nucleic acid targets is by quantification of fluorescence from the hybridised fluorescently labelled nucleic acid. More preferably quantitation may be from a fluorescently labelled reagent that binds a hapten incorporated into the nucleic acid.

In a preferred embodiment of the invention analysis of hybridisation may be achieved using suitable analysis software, such as the Microarray Analysis Suite (Affymetrix Inc.) and prognosis automated by use of classification software (for example Partek Genomics Suite from Partek Inc).

Effective quantification may be achieved using a fluorescence microscope which can be equipped with an automated stage to permit automatic scanning of the array, and which can be equipped with a data acquisition system for the automated measurement, recording and subsequent processing of the fluorescence intensity information. Suitable arrangements for such automation are conventional and well known to those skilled in the art.

In a preferred embodiment, the hybridised nucleic acids are detected by detecting one or more detectable moieties attached to the nucleic acids. The detectable moieties may be incorporated by any of a number of means well known to those of skill in the art. However, in a preferred embodiment, such moieties are simultaneously incorporated during an amplification step in the preparation of the sample nucleic acids (probes or targets). Thus, for example, polymerase chain reaction (PCR) using primers or nucleotides labelled with a detectable moiety will provide an amplification product labelled with said moiety. In a preferred embodiment, transcription amplification using a fluorescently labelled nucleotide (e.g. fluorescein-labelled UTP and/or CTP) incorporates the label into the transcribed nucleic acids.

Alternatively, a suitable detectable moiety may be added directly to the original nucleic acid sample (e.g., mRNA, polyA mRNA, cDNA, etc. from the tissue of interest) or to an amplification product after amplification of the original nucleic acid is completed. Means of attaching labels such as fluorescent labels to nucleic acids are well known to those skilled in the art and include, for example nick translation or end-labelling (e.g. with a labeled RNA) by kinasing of the nucleic acid and subsequent attachment (ligation) of a nucleic acid linker joining the sample nucleic acid to a label (such as a suitable fluorophore).

As set out previously, in addition to the methods and kits described above, the invention also provides a kit for determining susceptibility to keloid formation, the kit comprising:

i) at least one probe capable of binding specifically to a target molecule representative of expression in a patient of at least one gene selected from the group set out in Table 1; and
ii) reference material able to indicate the level of expression of said at least one gene in a control sample.

Preferably kits in accordance with this aspect of the invention may further comprise assay control material able to indicate that an assay has been performed correctly. Suitably such assay control material may include target molecules representative of expression of genes the expression of which does not vary between keloid and non-keloid tissues. Suitable examples of such housekeeping genes are considered elsewhere in the specification, and target molecules representative of expression of any of these genes may be advantageously provided in the kits of the invention. The provision of housekeeping genes of this sort in known quantities may provide a “standard” against which assay results may be normalised.

It may be preferred that a kit according to the present invention further comprises material (such as target molecules) representative of one or more genes whose expression increases in association with an increased likelihood of keloid formation. The provision of such genes may increase the ability to discriminate a biologically meaningful result from a change in the absolute input material or a change in the efficiency of any assay process. For example, lysyl oxidase displays a 5-fold higher expression in tissue associated with an increased likelihood of keloid formation. Lysyl oxidase is a key enzyme involved in collagen cross-linking and has previously been shown to be highly expressed in fibrotic tissue.

Kits of the invention may further comprise materials for the preparation of a population of target molecules representative of gene expression in a patient (or control tissue). Such materials may be suitable for the preparation of a population of nucleic acid target molecules. Alternatively such materials may be suitable for the preparation of a population of protein target molecules. It may be preferred that the kits comprise materials for the preparation of a population of labelled target molecules representative of gene expression in a patient or control tissue.

It is also preferred that kits of the present invention may further comprise an algorithm or reference data/material able to indicate that the level of expression of said at least one gene, selected from the group set out in Table 1, in the patient's sample is prognostic for keloid formation.

The algorithm may be provided in the form of a mathematical model of the difference in gene expression of said at least one gene, selected from the group set out in Table 1, between control and patient data (such as known patient data). This mathematical model may then be deployed on gene expression data of said at least one gene, selected from the group set out in Table 1, from a new patient sample. The output thus generated will thus provide a prediction of keloid prognosis.

Probes for inclusion in kits in accordance with this second aspect of the invention may be selected using the same criteria as for the first aspect of the invention. Suitable probes may be selected from the group comprising oligonucleotide probes, antibodies, aptamers and specific binding proteins.

Kits in accordance with the present invention may preferably comprise probes capable of binding specifically to target molecules representative of expression of up to five genes selected from the group set out in Table 1 (i.e. target molecules representative of the expression of up to five genes selected from Table 1). It is particularly preferred that kits of the invention comprise probes capable of binding 5, 6, 7, 8, 9 or 10 such target molecules. Suitable kits may comprise probes capable of binding to up to 15, 20, 30, 40 or 50 such target molecules. Indeed, kits of the invention may comprise probes capable of binding specifically to 50 or more target molecules, and may even comprise probes capable of binding specifically to targets representative of expression of all 55 of the genes set out in Table 1.

A kit of the invention will comprise probes capable of binding to target molecules representative of expression of at least one gene selected from Table 1, may preferably comprise probes capable of binding to target molecules representative of expression of at least one gene selected from Table 2, and may even more preferably comprise probes capable of binding to target molecules representative of expression of at least one gene selected from Table 3.

The probes provided in the kits of the invention may preferably be labelled probes. Labelled probes may comprise any detectable moiety considered in connection with the first aspect of the invention. Preferred labelled probes may be chosen from the group comprising haptens, fluorescently labelled probes, radioactively labelled probes and enzymatically labelled probes.

The reference material provided in kits of the invention may comprise a library of nucleic acid targets representative of expression in an appropriate control sample of one or more genes selected from the group of genes set out in Table 1.

In a preferred embodiment the reference material may comprise recorded information regarding the level of expression of one or more genes selected from the group of genes set out in Table 1 in keloid forming and non-keloid forming tissue

In a most preferred example the reference data may be used to create an algorithm which may deliver a prognosis based upon the level of expression of one or more genes selected from the group of genes set out in Table 1.

Oligonucleotide probes provided in kits of the invention, may preferably be provided in the form of an oligonucleotide array as considered elsewhere in the specification.

It will be appreciated from the preceding pages that the use of oligonucleotide arrays is particularly useful in the determination in accordance with the present invention of a patient's susceptibility to keloid formation.

Accordingly, in a third aspect of the invention there is provided an array of oligonucleotide probes, characterised in that at least 0.44% of the oligonucleotides probes present in the array are representative of genes selected from the group of genes set out in Table 1.

The invention also provides an array comprising immobilized antibody probes capable of binding specifically to molecules representative of expression of one or more of the group of genes set out in Table 1. Furthermore, the invention also provides an array comprising a nylon substrate to which are adhered nucleic acid probes representative of genes selected from the group of genes set out in Table 1. The nucleic acid probes may preferably be cDNA molecules.

Although a planar array surface is preferred, the array may be fabricated on a surface of virtually any shape or even a multiplicity of surfaces. In a further example a suitable array may be fabricated on the surface of a library of addressable beads, in which each bead displays a known nucleic acid sequence. Alternatively, a suitable array may be fabricated on the surface of a nylon substrate, typically a woven or non-woven nylon membrane.

It will be appreciated that arrays in accordance with the present invention can be used to compare the expression of a large number of genes set out in Table 1 simultaneously (and indeed to compare simultaneous expression of such genes), and that this gives rise to significant advantages in reduced labour, cost and time. Furthermore, the comparison of expression levels of multiple genes allows a greater degree of confidence in the determination of susceptibility to keloid formation.

An array in accordance with the present invention may comprise up to five probes specific for genes selected from the group set out in Table 1. Preferably an array may comprise 5, 6, 7, 8, 9 or ten probes specific for genes selected from the group set out in Table 1. Suitable arrays may comprise up to 15, up to 20, up to 30, up to 40 or up to 50 probes specific genes selected from the group set out in Table 1. Indeed, suitable arrays may comprise probes specific for 50 or more (and up to 55) of the genes set out in Table 1. It will be appreciated that each of the probes should be specific for a different selected gene, and that more than one copy of each probe may be provided.

Arrays of the invention may comprise one or more genes set out in Table 2 and/or, one or more genes set out in Table 3.

An array in accordance with the present invention may preferably comprise at least one gene from the group set out in Table 2, more preferably at least one gene from the group set out in Table 3.

It is preferred that an array according to the present invention may further comprise one or more genes whose expression increases in association with an increased likelihood of keloid formation. The provision of such genes may increase the ability to discriminate a biologically meaningful result from a change in the absolute input material or a change in the efficiency of any assay process. For example, lysyl oxidase displays a 5-fold higher expression in tissue associated with an increased likelihood of keloid formation. Lysyl oxidase is a key enzyme involved in collagen cross-linking and has previously been shown to be highly expressed in fibrotic tissue.

The methods, kits and arrays of the invention may also make use of one or more “housekeeping genes” to provide a control by which the efficiency of any assay may be assessed. These housekeeping genes may be provided in the kits of the invention, or on the arrays of the invention. Suitable housekeeping genes will be those that are either invariant or unassociated with keloid formation. Examples of genes that display invariant expression in both keloid and non-keloid (control) biopsy samples include exportin 7 (XPO7), Cleavage and Polyadenylation Specific Factor 4, 30 kDa (CPSF4), F-box only protein 7 (FBXO7), ADP-ribosylation factor 1 (ARF1), signal sequence receptor beta (SSR2) and methionine-tRNA synthetase (MARS).

Oligonucleotide arrays in accordance with the invention may be synthesized by any suitable technique known in the art. A preferred technique that may be used in the synthesis of such arrays is light-directed very large scaled immobilized polymer synthesis (VLSIPS), which has previously been described in a number of publications (Lipshutz R J, Fodor S P, Gingeras T R, Lockhart D J. High density synthetic oligonucleotide arrays. Nat Genet. 1999 January; 21(1 Suppl):20-4; Jacobs J W, Fodor S P. Combinatorial chemistry—applications of light-directed chemical synthesis. Trends Biotechnol. 1994 January; 12(1):19-26).

An oligonucleotide array in accordance with the invention may allow comparison of hybridisation, and thereby gene expression, to be carried out in extremely small fluid volumes (e.g., 250 μl or less, more preferably 100 μl or less, and most preferably 10 μl or less). This confers a number of advantages. In small volumes, hybridization may proceed very rapidly. In addition, hybridization conditions are extremely uniform throughout the sample, and the hybridization format is amenable to automated processing.

### Table Legends

Genes the expression of which may be investigated in accordance with the present invention are set out in the accompanying Tables. These Tables provide, in respect of each gene, a Gene Identification Number; a Public Identifier and Data Source (by which the skilled person may identify the gene in question and obtain further information regarding its sequence); the Gene Name; a Probe ID (setting out details of at least one probe that may be used to investigate expression of the gene in question); details of tissues that may be used in comparing expression of the gene in question; as well as details of the Fold Change in expression and P value derived from comparisons conducted as described in the Experimental Results section.

Table 1: Genes, the decreased expression of which in a tissue of interest versus a control sample, is indicative of increased susceptibility to keloid formation. All genes are highly statistically significant with p-values less than 0.01.

Table 2: Genes, the decreased expression of which in a tissue of interest versus a control sample, is indicative of increased susceptibility to keloid formation. All genes are highly statistically significant with p-values less than 0.01. All genes display a greater than 1.5-fold decreased expression in keloid-susceptible tissue.

Table 3: Genes, the decreased expression of which in a tissue of interest versus a control sample, is indicative of increased susceptibility to keloid formation. All genes are highly statistically significant with p-values less than 0.01. All genes display a greater than 2-fold decreased expression in keloid-susceptible tissue.

The invention will now be described further, with reference to the following Experimental Results.

### Experimental Results

The suitability of the genes set out in Table 1 for use in the determination of susceptibility to keloid formation is illustrated by the following study. In this study expression of the genes set out in Table 1 was compared between samples taken from known keloid tissues and suitably matched control tissues.

### 1.1 Diagnosis of Keloid Tissue.

Four patients of the African Continental Ancestry Group who had keloids that had been established for at least one year provided keloid samples for use in the present study. Only keloids for which a full medical history could be established were included. The age of the scar, a thorough review of the scar history and examination by a clinician, ensured that the scar had been correctly diagnosed as keloidal and not hypertrophic.

Three subjects from the African Continental Ancestry Group with no history of keloid formation provided control tissue material for use in the study described herein.

### 1.2 Tissue Collection.

Keloids were sampled using ellipsoid excisions perpendicular to the keloid margin and the resulting biopsies were sectioned to provide samples comprising the skin bordering the keloid (extra-lesional tissue). Since keloids tend to expand beyond the boundaries of the initial lesion this extra-lesional tissue provides an experimental example of a tissue predisposed to keloid formation.

Skin tissue from non-keloid forming individuals was biopsied in a similar manner to provide relevant control tissues.

Once collected, the biopsy sections were immersed in RNA Later solution (Ambion) and stored at −80° C. until required for later analysis of gene expression.

### 1.3 Preparation of Samples Representative of Gene Expression

Extra-lesional tissue samples from keloid formers and skin samples from non-keloid formers were disrupted using a Diax (G-10) homogeniser in the presence of proprietary Qiagen lysis buffer, and the lysate produced then incubated with proteinase K at 55° C. for 20 minutes.

Following incubation the mixture was separated by centrifugation, and RNA present purified using a RNeasy midi spin column (Qiagen Ltd).

### 1.4 Production of Nucleic Acid Targets.

10 μg total RNA (extracted from skin samples from both keloid and non-keloid formers) was used as substrate for cDNA synthesis using the Superscript System (Invitrogen Corp.). The resulting cDNA was then converted to biotinylated cRNA target molecules using the BioArray RNA Transcript labelling Kit (Enzo Life Sciences Inc.). The cRNA target molecules were subsequently purified from the reaction mixture using a RNeasy mini kit (Qiagen Ltd). 20 μg cRNA was fragmented for array hybridisation.

### 1.5 Comparison of Gene Expression.

Fragmented cRNA target molecules representative of gene expression in extra-lesional tissues predisposed to keloid formation and in control tissue not predisposed to keloid formation, were hybridised to oligonucleotide arrays comprising oligonucleotide probes representing the genes set out in Table 1. Standard Affymetrix protocols (Affymetrix Inc) were used to effect hybridisation. The hybridised arrays were stained with streptavidin-phycoerythrin and then scanned using a laser confocal scanner to generate fluorescence intensities.

All arrays were normalised to a target intensity of 1000, and signal values and detection P-values were calculated using the Microarray Analysis Suite version 5.0 software. Data sets passing quality control were imported into the Spotfire analysis suite for comparison of expression with that in control tissues.

Signal values were transformed to log 2 scale and t-tests, comparing the gene expression in samples representative of keloids with expression in controls, were performed on the log 2 transformed data. Mean signal values were calculated for each sample group and fold changes were calculated from these mean values.

### 1.6 Results.

T-tests comparing expression of the genes set out in Table 1 in tissues predisposed to keloid formation with expression of the same genes in control tissues not predisposed to keloid formation all had a t-test p-value of less than 0.01. This confirms that the expression of each and all of the genes set out in Table 1 are highly significantly decreased in tissues predisposed to keloid formation as compared to controls.

These results clearly illustrate that decreased expression in a sample from a patient of one or more genes from the group set out in Table 1, as compared to expression of the same gene or genes in a control sample, provides a clear indication that the patient is susceptible to keloid formation.

TABLE 1
Prognostic Down

| Gene | Public | | | | | Fold | |
|---|---|---|---|---|---|---|---|
| ID | Identifier | Data Source | Gene Name | Probe_ID | Comparison | Change | P value |
| 1 | 3119 | Entrez Gene | major histocompatibility complex, class II, DQ | 36878_f_at | Day 7 Extra/Day 7 Control | 0.35 | 3.4504E−03 |
| | | | beta 1 | | | | |
| 2 | AC002045 | GenBank | nuclear pore complex interacting protein /// | 33836_at | Day 3 Extra/Day 3 Control | 0.36 | 5.4761E−03 |
| | | | KIAA0220-like protein /// hypothetical gene | | | | |
| | | | LOC283846 /// hypothetical protein | | | | |
| | | | LOC283970 /// hypothetical | | | | |
| 3 | 3034 | Entrez Gene | histidine ammonia-lyase | 40735_at | Day 3 Extra/Day 3 Control | 0.45 | 1.3220E−03 |
| 4 | 3709 | Entrez Gene | Family with sequence similarity 20, member C | 33954_at | Day 7 Extra/Day 3 Extra | 0.49 | 5.0454E−03 |
| 5 | 9349 | Entrez Gene | ribosomal protein L23 | 32395_r_at | Day 7 Extra/Day 3 Extra | 0.50 | 8.7494E−03 |
| 6 | 10439 | Entrez Gene | olfactomedin 1 | 36134_at | Day 7 Extra/Day 3 Extra | 0.52 | 2.1097E−03 |
| 7 | 7138 | Entrez Gene | troponin T type 1 (skeletal, slow) | 36113_s_at | Day 7 Extra/Day 7 Control | 0.52 | 6.2685E−03 |
| 8 | 2995 | Entrez Gene | glycophorin C (Gerbich blood group) | 38119_at | Day 7 Extra/Day 7 Control | 0.52 | 2.0937E−04 |
| 9 | 10724 | Entrez Gene | meningioma expressed antigen 5 (hyaluronidase) | 35317_at | Day 3 Extra/Day 3 Control | 0.53 | 5.6964E−03 |
| 10 | 10781 | Entrez Gene | zinc finger protein 266 | 41621_i_at | Day 3 Extra/Day 3 Control | 0.53 | 3.9035E−03 |
| 11 | HG3521- | The Institute | — | 1903_at | Day 3 Extra/Day 3 Control | 0.53 | 4.8991E−03 |
| | HT371 | for Genomic | | | | | |
| | | Research | | | | | |
| 12 | 9445 | Entrez Gene | Integral membrane protein 2B | 41301_at | Day 7 Extra/Day 3 Extra | 0.53 | 9.3140E−03 |
| 13 | 9796 | Entrez Gene | phytanoyl-CoA hydroxylase interacting protein | 37191_at | Day 7 Extra/Day 7 Control | 0.55 | 2.4313E−03 |
| | | | | 37191_at | Day 7 Extra/Day 3 Extra | 0.68 | 9.9783E−03 |
| 14 | 51526 | Entrez Gene | chromosome 20 open reading frame 111 | 36934_at | Day 3 Extra/Day 3 Control | 0.56 | 1.4204E−03 |
| 15 | 80308 | Entrez Gene | Fad1, flavin adenine dinucleotide synthetase, | 39074_at | Day 3 Extra/Day 3 Control | 0.58 | 6.9762E−03 |
| | | | homolog (yeast) | | | | |
| 16 | 25906 | Entrez Gene | DKFZP564M082 protein | 35715_at | Day 7 Extra/Day 7 Control | 0.58 | 5.3129E−03 |
| 17 | 9682 | Entrez Gene | Jumonji domain containing 2A | 32073_at | Day 3 Extra/Day 3 Control | 0.60 | 8.6112E−03 |
| 18 | 3615 | Entrez Gene | IMP (inosine monophosphate) dehydrogenase 2 | 36624_at | Day 7 Extra/Day 3 Extra | 0.60 | 7.9800E−04 |
| | | | | 36624_at | Day 7 Extra/Day 7 Control | 0.72 | 7.8494E−03 |
| 19 | 4681 | Entrez Gene | neuroblastoma, suppression of tumorigenicity 1 | 37005_at | Day 7 Extra/Day 7 Control | 0.60 | 2.4458E−03 |
| 20 | 2954 | Entrez Gene | glutathione transferase zeta 1 (maleylacetoacetate | 1212_at | Day 7 Extra/Day 3 Extra | 0.61 | 6.1710E−03 |
| | | | isomerase) | | | | |
| 21 | 113791 | Entrez Gene | HGFL gene | 36231_at | Day 3 Extra/Day 3 Control | 0.62 | 6.3175E−03 |
| 22 | 5939 | Entrez Gene | RNA binding motif, single stranded interacting | 34187_at | Day 7 Extra/Day 3 Extra | 0.63 | 9.5160E−04 |
| | | | protein 2 | | | | |
| 23 | HG3914- | The Institute | — | 1790_s_at | Day 3 Extra/Day 3 Control | 0.63 | 2.4941E−03 |
| | HT418 | for Genomic | | | | | |
| | | Research | | | | | |
| 24 | 25937 | Entrez Gene | WW domain containing transcription regulator 1 | 33876_at | Day 3 Extra/Day 3 Control | 0.64 | 8.6885E−03 |
| 25 | 4154 | Entrez Gene | muscleblind-like (Drosophila) | 34306_at | Day 3 Extra/Day 3 Control | 0.64 | 4.6013E−03 |
| 26 | 3275 | Entrez Gene | HMT1 hnRNP methyltransferase-like 1 | 39348_at | Day 7 Extra/Day 7 Control | 0.64 | 1.9896E−04 |
| | | | (S. cerevisiae) | | | | |
| 27 | 8667 | Entrez Gene | eukaryotic translation initiation factor 3, subunit 3 | 35327_at | Day 7 Extra/Day 7 Control | 0.65 | 7.1558E−03 |
| | | | gamma, 40 kDa | | | | |
| 28 | 23277 | Entrez Gene | KIAA0664 protein | 34259_at | Day 7 Extra/Day 3 Extra | 0.66 | 4.3005E−03 |
| 29 | 8673 | Entrez Gene | vesicle-associated membrane protein 8 | 32715_at | Day 7 Extra/Day 3 Extra | 0.67 | 3.9499E−03 |
| | | | (endobrevin) | | | | |
| 30 | 1540 | Entrez Gene | Cylindromatosis (turban tumor syndrome) | 39582_at | Day 3 Extra/Day 3 Control | 0.68 | 4.8831E−03 |
| 31 | 9776 | Entrez Gene | KIAA0652 gene product | 38020_at | Day 3 Extra/Day 3 Control | 0.68 | 9.2768E−03 |
| 32 | 1203 | Entrez Gene | ceroid-lipofuscinosis, neuronal 5 | 34324_at | Day 3 Extra/Day 3 Control | 0.68 | 7.9955E−03 |
| 33 | 1632 | Entrez Gene | dodecenoyl-Coenzyme A delta isomerase | 37982_at | Day 7 Extra/Day 3 Extra | 0.68 | 4.3631E−03 |
| | | | (3,2 trans-enoyl-Coenzyme A isomerase) | | | | |
| 34 | 51304 | Entrez Gene | zinc finger, DHHC-type containing 3 | 39751_at | Day 7 Extra/Day 3 Extra | 0.68 | 1.4164E−03 |
| 35 | 516 | Entrez Gene | ATP synthase, H+ transporting, mitochondrial | 38076_at | Day 7 Extra/Day 3 Extra | 0.69 | 6.1895E−03 |
| | | | F0 complex, subunit c (subunit 9), isoform, 1 | | | | |
| 36 | 10899 | Entrez Gene | jumping translocation breakpoint | 41833_at | Day 3 Extra/Day 3 Control | 0.69 | 5.3833E−03 |
| 37 | 171546 | Entrez Gene | chromosome 14 open reading frame 147 | 33399_at | Day 7 Extra/Day 3 Extra | 0.70 | 2.2549E−03 |
| 38 | 10939 | Entrez Gene | AFG3 ATPase family gene 3-like 2 (yeast) | 34315_at | Day 7 Extra/Day 3 Extra | 0.70 | 1.0600E−04 |
| 39 | 1933 | Entrez Gene | eukaryotic translation elongation factor 1 beta 2 | 35748_at | Day 7 Extra/Day 3 Extra | 0.70 | 8.5569E−03 |
| 40 | 5379 | Entrez Gene | postmeiotic segregation increased 2-like 1 | 178_f_at | Day 3 Extra/Day 3 Control | 0.71 | 7.4507E−03 |
| 41 | 79073 | Entrez Gene | hypothetical protein MGC5508 | 39693_at | Day 7 Extra/Day 3 Extra | 0.72 | 8.8935E−03 |
| 42 | 9139 | Entrez Gene | core-binding factor, runt domain, alpha subunit 2; | 40050_at | Day 3 Extra/Day 3 Control | 0.72 | 2.4234E−03 |
| | | | translocated to, 2 | | | | |
| 43 | 9202 | Entrez Gene | zinc finger protein 262 | 39762_at | Day 3 Extra/Day 3 Control | 0.72 | 3.9310E−03 |
| 44 | 2592 | Entrez Gene | galactose-1-phosphate uridylyltransferase | 36664_at | Day 7 Extra/Day 3 Extra | 0.73 | 6.8009E−03 |
| 45 | 56339 | Entrez Gene | Methyltransferase like 3 | 32244_at | Day 7 Extra/Day 3 Extra | 0.73 | 4.9666E−03 |
| 46 | 6626 | Entrez Gene | small nuclear ribonucleoprotein polypeptide A | 40842_at | Day 7 Extra/Day 3 Extra | 0.73 | 5.1288E−04 |
| 47 | AI341574 | GenBank | postmeiotic segregation increased 2-like 1 /// | 32310_f_at | Day 3 Extra/Day 3 Control | 0.74 | 8.3897E−03 |
| | | | postmeiotic segregation increased 2-like 5 /// | | | | |
| | | | similar to postmeiotic segregation | | | | |
| | | | increased 2-like 2 // | | | | |
| 48 | 10980 | Entrez Gene | COP9 constitutive photomorphogenic homolog | 40138_at | Day 7 Extra/Day 7 Control | 0.75 | 7.9786E−03 |
| | | | subunit 6 (Arabidopsis) | | | | |
| 49 | 9092 | Entrez Gene | squamous cell carcinoma antigen recognised by | 33706_at | Day 7 Extra/Day 3 Extra | 0.76 | 5.4076E−03 |
| | | | T cells | | | | |
| 50 | 25864 | Entrez Gene | abhydrolase domain containing 14A | 41018_at | Day 7 Extra/Day 7 Control | 0.76 | 8.5607E−03 |
| 51 | 6746 | Entrez Gene | signal sequence receptor, beta | 36147_at | Day 7 Extra/Day 7 Control | 0.77 | 8.2523E−03 |
| | | | (translocon-associated protein beta) | | | | |
| 52 | 2621 | Entrez Gene | growth arrest-specific 6 | 37658_at | Day 3 Extra/Day 3 Control | 0.78 | 7.2186E−03 |
| 53 | HG1980- | The Institute | — | 956_at | Day 7 Extra/Day 3 Extra | 0.83 | 7.7875E−03 |
| | HT202 | for Genomic | | | | | |
| | | Research | | | | | |
| 54 | 56948 | Entrez Gene | chromosome 14 open reading frame 124 | 32591_at | Day 3 Extra/Day 3 Control | 0.84 | 5.8019E−03 |
| 55 | 35 | Entrez Gene | acyl-Coenzyme A dehydrogenase, C-2 to C-3 | 39408_at | Day 3 Extra/Day 3 Control | 0.90 | 8.7720E−03 |
| | | | short chain | | | | |

TABLE 2
Prognostic Down

| Gene | Public | | | | | Fold | |
|---|---|---|---|---|---|---|---|
| ID | Identifier | Data Source | Gene Name | Probe_ID | Comparison | Change | P value |
| 1 | 3119 | Entrez Gene | major histocompatibility complex, class II, DQ | 36878_f_at | Day 7 Extra/Day 7 Control | 0.35 | 3.4504E−03 |
| | | | beta 1 | | | | |
| 2 | AC002045 | GenBank | nuclear pore complex interacting protein /// | 33836_at | Day 3 Extra/Day 3 Control | 0.36 | 5.4761E−03 |
| | | | KIAA0220-like protein /// hypothetical gene | | | | |
| | | | LOC283846 /// hypothetical | | | | |
| | | | protein LOC283970 /// hypothetical | | | | |
| 3 | 3034 | Entrez Gene | histidine ammonia-lyase | 40735_at | Day 3 Extra/Day 3 Control | 0.45 | 1.3220E−03 |
| 4 | 3709 | Entrez Gene | Family with sequence similarity 20, member C | 33954_at | Day 7 Extra/Day 3 Extra | 0.49 | 5.0454E−03 |
| 5 | 9349 | Entrez Gene | ribosomal protein L23 | 32395_r_at | Day 7 Extra/Day 3 Extra | 0.50 | 8.7494E−03 |
| 6 | 10439 | Entrez Gene | olfactomedin 1 | 36134_at | Day 7 Extra/Day 3 Extra | 0.52 | 2.1097E−03 |
| 7 | 7138 | Entrez Gene | troponin T type 1 (skeletal, slow) | 36113_s_at | Day 7 Extra/Day 7 Control | 0.52 | 6.2685E−03 |
| 8 | 2995 | Entrez Gene | glycophorin C (Gerbich blood group) | 38119_at | Day 7 Extra/Day 7 Control | 0.52 | 2.0937E−04 |
| 9 | 10724 | Entrez Gene | meningioma expressed antigen 5 (hyaluronidase) | 35317_at | Day 3 Extra/Day 3 Control | 0.53 | 5.6964E−03 |
| 10 | 10781 | Entrez Gene | zinc finger protein 266 | 41621_i_at | Day 3 Extra/Day 3 Control | 0.53 | 3.9035E−03 |
| 11 | HG3521- | The Institute | — | 1903_at | Day 3 Extra/Day 3 Control | 0.53 | 4.8991E−03 |
| | HT371 | for Genomic | | | | | |
| | | Research | | | | | |
| 12 | 9445 | Entrez Gene | Integral membrane protein 2B | 41301_at | Day 7 Extra/Day 3 Extra | 0.53 | 9.3140E−03 |
| 13 | 9796 | Entrez Gene | phytanoyl-CoA hydroxylase interacting protein | 37191_at | Day 7 Extra/Day 7 Control | 0.55 | 2.4313E−03 |
| | | | | 37191_at | Day 7 Extra/Day 3 Extra | 0.68 | 9.9783E−03 |
| 14 | 51526 | Entrez Gene | chromosome 20 open reading frame 111 | 36934_at | Day 3 Extra/Day 3 Control | 0.56 | 1.4204E−03 |
| 15 | 80308 | Entrez Gene | Fad1, flavin adenine dinucleotide synthetase, | 39074_at | Day 3 Extra/Day 3 Control | 0.58 | 6.9762E−03 |
| | | | homolog (yeast) | | | | |
| 16 | 25906 | Entrez Gene | DKFZP564M082 protein | 35715_at | Day 7 Extra/Day 7 Control | 0.58 | 5.3129E−03 |
| 17 | 9682 | Entrez Gene | jumonji domain containing 2A | 32073_at | Day 3 Extra/Day 3 Control | 0.60 | 8.6112E−03 |
| 18 | 3615 | Entrez Gene | IMP (inosine monophosphate) dehydrogenase 2 | 36624_at | Day 7 Extra/Day 3 Extra | 0.60 | 7.9800E−04 |
| | | | | 36624_at | Day 7 Extra/Day 7 Control | 0.72 | 7.8494E−03 |
| 19 | 4681 | Entrez Gene | neuroblastoma, suppression of tumorigenicity 1 | 37005_at | Day 7 Extra/Day 7 Control | 0.60 | 2.4458E−03 |
| 20 | 2954 | Entrez Gene | glutathione transferase zeta 1 (maleylacetoacetate | 1212_at | Day 7 Extra/Day 3 Extra | 0.61 | 6.1710E−03 |
| | | | isomerase) | | | | |
| 21 | 113791 | Entrez Gene | HGFL gene | 36231_at | Day 3 Extra/Day 3 Control | 0.62 | 6.3175E−03 |
| 22 | 5939 | Entrez Gene | RNA binding motif, single stranded interacting | 34187_at | Day 7 Extra/Day 3 Extra | 0.63 | 9.5160E−04 |
| | | | protein 2 | | | | |
| 23 | HG3914- | The Institute | — | 1790_s_at | Day 3 Extra/Day 3 Control | 0.63 | 2.4941E−03 |
| | HT418 | for Genomic | | | | | |
| | | Research | | | | | |
| 24 | 25937 | Entrez Gene | WW domain containing transcription regulator 1 | 33876_at | Day 3 Extra/Day 3 Control | 0.64 | 8.6885E−03 |
| 25 | 4154 | Entrez Gene | muscleblind-like (Drosophila) | 34306_at | Day 3 Extra/Day 3 Control | 0.64 | 4.6013E−03 |
| 26 | 3275 | Entrez Gene | HMT1 hnRNP methyltransferase-like 1 | 39348_at | Day 7 Extra/Day 7 Control | 0.64 | 1.9896E−04 |
| | | | (S. cerevisiae) | | | | |
| 27 | 8667 | Entrez Gene | eukaryotic translation initiation factor 3, | 35327_at | Day 7 Extra/Day 7 Control | 0.65 | 7.1558E−03 |
| | | | subunit 3, gamma, 40 kDa | | | | |
| 28 | 23277 | Entrez Gene | KIAA0664 protein | 34259_at | Day 7 Extra/Day 3 Extra | 0.66 | 4.3005E−03 |

TABLE 3
Prognostic Down

| Gene | Public | | | | | Fold | |
|---|---|---|---|---|---|---|---|
| ID | Identifier | Data Source | Gene Name | Probe_ID | Comparison | Change | P value |
| 1 | 3119 | Entrez Gene | major histocompatibility complex, class II DQ | 36878_f_at | Day 7 Extra/Day 7 Control | 0.35 | 3.4504E−03 |
| | | | beta 1 | | | | |
| 2 | AC002045 | GenBank | nuclear pore complex interacting protein /// | 33836_at | Day 3 Extra/Day 3 Control | 0.36 | 5.4761E−03 |
| | | | KIAA0220-like protein /// hypothetical gene | | | | |
| | | | LOC283846 /// hypothetical protein | | | | |
| | | | LOC283970 /// hypothetical | | | | |
| 3 | 3034 | Entrez Gene | histidine ammonia-lyase | 40735_at | Day 3 Extra/Day 3 Control | 0.45 | 1.3220E−03 |
| 4 | 3709 | Entrez Gene | Family with sequence similarity 20, member C | 33954_at | Day 7 Extra/Day 3 Extra | 0.49 | 5.0454E−03 |
| 5 | 9349 | Entrez Gene | ribosomal protein L23 | 32395_r_at | Day 7 Extra/Day 3 Extra | 0.50 | 8.7494E−03 |

1. A method for determining susceptibility to keloid formation, the method comprising: comparing expression, in a sample representative of gene expression in a patient, of at least one gene, selected from the group of genes set out in Table 1, with expression of the said at least one gene in a control sample; wherein decreased expression of said at least one gene in the sample representative of gene expression in the patient compared to expression of said at least one gene in the control sample indicates that the patient is susceptible to keloid formation.

2. A method according to claim 1, wherein the method is an in vitro method.

3. A method according to claim 1, comprising comparing the expression of at least one gene selected from the group of genes set out in Table 2.

4. A method according to claim 1, comprising comparing the expression of at least one gene selected from the group of genes set out in Table 3.

5. A method according to claim 1, wherein the sample representative of gene expression in the tissue of interest comprises a nucleic acid target molecule.

6. A method according to claim 5, wherein the nucleic acid target molecule comprises an RNA oligonucleotide.

7. A method according to claim 5, wherein the nucleic acid target molecule comprises a DNA oligonucleotide.

8. A method according to claim 1, wherein the sample representative of gene expression in the tissue of interest comprises a protein target molecule.

9. A method according to claim 5, wherein the comparison of gene expression is effected using a probe molecule capable of binding specifically to the target molecule.

10. A method according to claim 9, wherein the probe molecule is selected from the group comprising oligonucleotide probes, antibodies and aptamers.

11. A method according to claim 1, wherein expression in the sample and expression in the control tissue is compared for at least 5 genes.

12. A method according to claim 1, wherein expression in the sample and expression in the control tissue is compared for between 5 and 10 genes.

13. A kit for determining susceptibility to keloid formation, the kit comprising: i) at least one probe capable of binding specifically to a target molecule representative of expression in the tissue of interest of at least one gene selected from the group set out in Table 1; and ii) reference material able to indicate the level of expression of said at least one gene in control tissue.

14. A kit according to claim 13, wherein the probe comprises an oligonucleotide probe.

15. A kit according to claim 13, wherein the probe comprises an antibody.

16. A kit according to claim 13, wherein the probe comprises an aptamer.

17. A kit according to claim 13, wherein the probe is a labelled probe.

18. A kit according to claim 17, wherein the probe is a fluorescent-labelled probe.

19. A kit according to claim 17, wherein the probe is an enzyme-labelled probe.

20. A kit according to claim 17, wherein the probe is a radioactive-labelled probe.

21. A kit according to claim 13, comprising probes capable of binding specifically to target molecules representative of expression of at least 5 genes selected from the group set out in Table 1.

22. A kit according to claim 13, comprising probes capable of binding specifically to target molecules representative of expression of between 5 and 10 genes selected from the group set out in Table 1.

23. A kit according to any one of claim 13, wherein the reference material comprises a library of nucleic acid targets representative of expression of said at least one gene selected from the group of genes set out in Table 1.

24. A kit according to any one of claim 13, wherein the reference material comprises a library of protein targets representative of expression of said at least one gene selected from the group of genes set out in Table 1.

25. A kit according to any one of claim 13, wherein the reference material comprises data as to the expression of said at least one gene selected from the group of genes set out in Table 1.

26. A kit according to any one of claim 13, further comprising a prognostic algorithm.

27. A kit according to any one of claim 13, further comprising assay control material able to indicate that an assay has been performed correctly.

28. A kit according to any one of claim 13, further comprising materials for the preparation of a population of target molecules representative of gene expression in a patient.

29. An array of oligonucleotide probes, characterised in that at least 0.44% of the oligonucleotides probes present in the array are selected from the group of genes set out in Table 1.

30. An array comprising a nylon substrate to which are adhered nucleic acid probes representative of genes selected from the group of genes set out in Table 1.

31. An array comprising immobilized antibody probes capable of binding specifically to molecules representative of expression of one or more of the group of genes set out in Table 1.

32. A method according to claim 8, wherein the comparison of gene expression is effected using a probe molecule capable of binding specifically to the target molecule.

33. A method according to claim 32, wherein the probe molecule is selected from the group comprising oligonucleotide probes, antibodies and aptamers.
